(19) United States
(12) Patent Application Publication
(10) Pub. No.: US 2021/0041339 A1
(43) Pub. Date: Feb. 11, 2021

(54) METHODS FOR DETECTION OF THE PERMEATION OF CHEMICAL WARFARE AGENTS THROUGH MEMBRANES

(71) Applicant: RUTGERS, THE STATE UNIVERSITY OF NEW JERSEY, New Brunswick, NJ (US)

(72) Inventors: Georgios Tsilomelekis, Lawrenceville, NJ (US); Jonathan Colon Ortiz, Somerville, MA (US); Alexander V. Neimark, Princeton, NJ (US)

(21) Appl. No.: 16/988,945

(22) Filed: Aug. 10, 2020

Related U.S. Application Data
(60) Provisional application No. 62/884,417, filed on Aug. 8, 2019.

(51) Int. Cl. G01N 15/08; G01J 3/44; G01N 33/00

(52) U.S. Cl. CPC G01N 15/0806; B82Y 30/00; G01N 33/0057; G01J 3/44

(57) ABSTRACT

Methods for the detection of the permeation of chemical compounds across a membrane are provided herein.

The present application claims priority to U.S. Provisional Application No. 62/884,417, filed Aug. 8, 2019. The entire disclosure of the foregoing application is incorporated by reference herein.

This invention was made with government support under Grant No. HDTRA1-14-1-0015 awarded by the Defense Threat Reduction Agency (DTRA) and Grant No. 1705825 awarded by the National Science Foundation. The government has certain rights in the invention.

### FIELD OF THE INVENTION

The present invention relates generally to devices and methods for the detection of the traversal of chemical compounds, such as chemical warfare agents, across a membrane.

### BACKGROUND OF THE INVENTION

Protective materials against chemical warfare agents (CWA), such as nuclear, biological and chemical (NBC) protective clothing, have been evolving from impermeable butyl rubber-based materials to permeable polyurethane foams mixed with activated carbon (Boopathi, et al., Open Textile J. (2008) 1:1-8). One of the challenges in designing protective wearable gear is the ability of the material to quickly permeate sweat and dissipate heat while blocking penetration of CWA through the protective material. Butyl rubber-based materials, such as earlier generations of NBC protective clothing, are excellent at blocking hazardous chemicals, since they are impermeable, but are not able to dissipate heat or sweat from the body causing serious heat strain (Heled, et al., Aviat. Space Envir. Md. (2004) 75(5):391-396). One of the most promising technological developments includes the Joint Service Lightweight Integrated Suit Technology (JSLIST), a multilayered semipermeable adsorptive textile, as it provides improved chemical protection, more mobility for the user, and heat stress reduction features (Lin, et al., The Evaluation of Thermal Comfort for CB Protective Garments, In International Conference on Environmental Ergonomics XII, Mekjavic, et al., Eds. Biomed: Piran, Slovenia, 2007; pp. 375-378). An alternative approach encompasses the potential incorporation of secondary components, such as adsorbents or catalysts, where the hazardous substances can either selectively adsorb or decompose as they diffuse through the membranes respectively. Towards this, popular adsorbents like activated carbon are known for having substantially large surface area to volume ratios of over 2600 m²g⁻¹, leading to adsorption capacities of over 950 mg of adsorbate per gram of adsorbent (Baysal, et al., J. Environ. Chem. Eng. (2018) 6(2):1702-1713). However, the hazardous substances were not decomposed, remaining a threat even when trapped in activated charcoal until clothing was laundered (Boopathi, et al., Open Textile J. (2008) 1:1-8). Among alternative protective materials, polymer electrolyte membranes (PEM) have been considered as they possess breathability, selective permeability, and agent protective properties (Lu, et al., J. Membrane Sci. (2008) 318(1-2):397-404; Jung, et al., J. Membrane Sci. (2010) 362(1-2):137-142; Rivin, et al., J. Phys. Chem. B (2004) 108(26):8900-8909). The properties of PEMs can be tailored by loading functional metal-oxide nanoparticles (MONP) using the in situ growth mechanism (Landers, et al., Angewandte Chemie-International Edition (2016) 55(38):11522-11527). Growing MONP within PEM substrates allows for easy scalability for mass production as membranes go through very simple steps in order to incorporate inorganic catalysts. The incorporation of MONP within PEM substrates provide enhanced mechanical stability (Homayoonfal, et al., Desalin. Water Treat. (2013) 51(16-18):3295-3316), along with the ability to hinder the permeation of harmful substances, thus providing chemical protection.

Understanding the behavior of CWA through barriers enables the designer to devise clever ways to control or even enhance the protective properties of the materials involved in the protective material. Therefore, transport properties of chemicals such as CWA through membrane or fabric systems are of paramount importance when designing protective clothing, as they determine the level of protection when exposed to these agents. Improved methods for detecting whether a membrane can inhibit transport of a chemical or CWA are needed.

### SUMMARY OF THE INVENTION

The present invention provides devices and methods for the in situ detection of the permeation of a test compound across a membrane. In certain embodiments, the method comprises a) adding the test compound to a first chamber of a permeation cell or device, wherein the permeation cell comprises a first chamber and a second chamber which are separated by the membrane; and b) detecting the presence of the test compound in the second chamber by a spectroscopic method. In a particular embodiment, the test compound is detected in the second chamber as a function of time, wherein an increase in the presence of test compound in the second chamber over time indicates that the membrane is permeable to the test compound.

### BRIEF DESCRIPTION OF THE DRAWINGS

FIG. 1A provides wide angle x-ray diffractograms for Nexar®-ZnO (bottom), Nafion®-ZnO (middle), and hexagonal-lattice bulk ZnO reference (top). FIG. 1B provides TEM micrographs of ZnO in Nafion®. FIG. 1C provides a graph of the ZnO aggregate size distribution in Nafion®. FIG. 1D provides TEM micrographs of ZnO in Nexar®. FIG. 1E provides a graph of ZnO aggregate size distribution in Nexar®.

FIG. 2A provides a schematic of an in situ Raman spectroscopy setup for a permeation cell. FIG. 2B provides Raman spectra for dimethyl methylphosphonate (DMMP) samples in solutions of varying concentrations: 1 vol. % DMMP, 3 vol. % DMMP, 5 vol. % DMMP, 10 vol. % DMMP, 25 vol. % DMMP, 100 vol. % DMMP. Inset shows a closer look at DMMP peak at 715 cm⁻¹. FIG. 2C provides a calibration curve for DMMP based on Raman spectra.

FIGS. 3A-3D provide DMMP spectral evolution at peak located at 715 cm⁻¹ as a function of time for DMMP permeation through: Nafion®-H+(FIG. 3A), Nafion®-ZnO (FIG. 3B), Nexar®-H+(FIG. 3C), and Nexar®-ZnO (FIG. 3D). FIG. 3E provides Raman spectra of DMMP in the receiving compartment as a function of time through: Nafion®-H+(top left), Nafion®-ZnO (top right), Nexar®-H+(bottom left), and Nexar®-ZnO (bottom right). Spectra collected every 30 minutes until 360 minutes, bottom to top spectra, respectively.

FIG. 4A provides a graph of the DMMP concentration in receiving compartment as a function of time. FIG. 4B shows the rearranged DMMP permeation as expressed in Equation 2. Nafion®-H⁺ (1), Nafion®-ZnO (2), Nexar®-H⁺ (3), and Nexar®-ZnO (4) are shown. Error bars are based on standard deviation from duplicate experiments.

FIGS. 5A and 5B provide schematics of permeation cells. FIG. 5C provides a schematic of a gas-phase permeation assembly. FIGS. 5D and 5E provide schematics of liquid phase permeation assemblies.

### DETAILED DESCRIPTION OF THE INVENTION

Reliable measurement of the permeability of membranes with respect to different chemical compounds is of paramount importance for the design of novel materials for selective separations, protective barriers against chemical warfare agents, and toxic industrial chemicals. Herein, an in situ spectroscopy (e.g., Raman) experimental setup is provided which allows the measurement—in real time—of the dynamics of permeation of a chemical compound (e.g., a toxin or chemical warfare agent) across a membrane (e.g., a polyelectrolyte membrane). Efficiency of the method is demonstrated on two types of commercial membranes, Nafion® 117 and Nexar® MD9200, modified with metal oxide nanoparticles, with the nerve agent surrogate/simulant dimethyl methylphosphonate. It was found that loading membranes with ZnO nanoparticles significantly reduces agent permeation, enhancing its protective capabilities against hazardous substances.

In accordance with the instant invention, methods of detecting the permeation of a test compound (e.g., a chemical compound such as, for example, a toxin or chemical warfare agent) through a membrane are provided. While the methods are generally described herein in the context of detecting the permeation of chemical warfare agents across a membrane, the methods can be used to detect the permeation of any chemical compound across any membrane. For example, the methods of the instant invention may be used to detect the permeation of separation membranes to chemical compounds. Such separation membranes include, without limitation, diffusion barriers/membranes in electrochemical devices, such as fuel cells.

The methods of the instant invention allow for the in situ determination of the permeability of a target membrane. The methods do not require the taking of multiple samples for later testing. Rather, the methods of the instant invention allow for the determination of the permeability of a target membrane in real time and in situ.

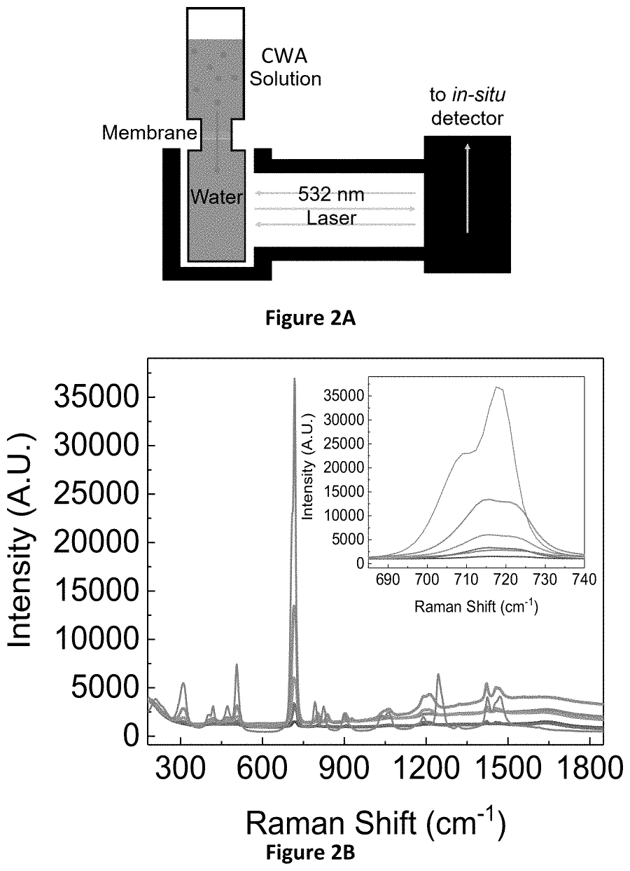

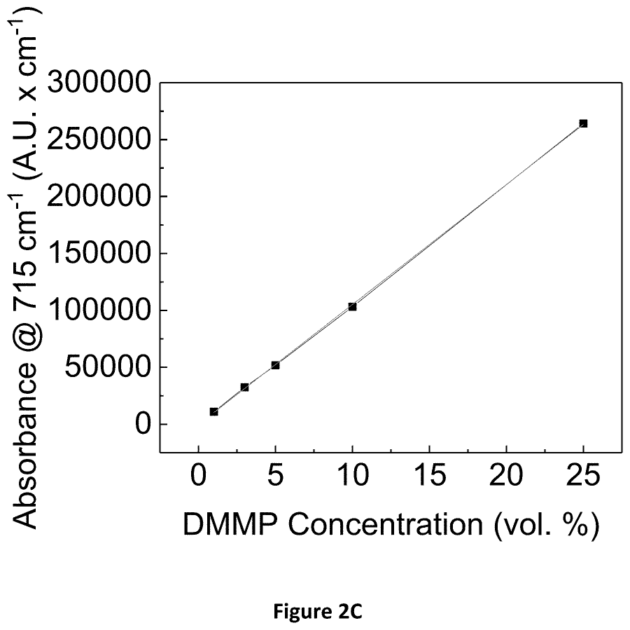

In a particular embodiment, the method comprises adding a test compound to a first chamber of a permeation cell and detecting the presence of the test compound in a second chamber (e.g., receiving chamber) of the permeation cell, wherein the first and second chambers are separated by a test membrane. FIG. 2A provides a schematic of an example permeation cell comprising a first and second chamber separated by a test membrane. The test membrane should be configured between the first and second chambers such that the only means for passage of a test compound from the first chamber to the second chamber is through the test membrane. In other words, the first and second chambers are physically separated by the test membrane. The first and second chambers of the permeation cell may be in gas and/or liquid connection and/or communication across the test membrane. The permeation cell, when fully assembled, is preferably hermetically sealed and/or airtight and prevents the escape of the test compound.

In a particular embodiment, the test membrane may seal (e.g., hermetically) the gap or space between the first and second chambers. In a particular embodiment, the test membrane covers or seals an opening or aperture of the first chamber and an opening or aperture of the second chamber. The opening or aperture of the first chamber may be the same size and shape as the opening or aperture of the second chamber. The opening or aperture of the first chamber may differ in shape and/or size from the opening or aperture of the second chamber so long as the test membrane physically separates the first and second chamber and the first and second chambers are in gas and/or liquid connection across the test membrane. For example, the opening or aperture of the first chamber may be larger than the opening or aperture of the second chamber but have the same shape such that the opening or aperture of the second chamber may be inserted into the opening or aperture of the first chamber with the test membrane between the two chambers and a seal being created around the test membrane between the two chambers. Alternatively, the opening or aperture of the second chamber may be larger than the opening or aperture of the first chamber but have the same shape such that the opening or aperture of the first chamber may be inserted into the opening or aperture of the second chamber with the test membrane between the two chambers and a seal being created around the test membrane between the two chambers. For example, the outer diameter of the opening or aperture of one chamber may approximate or be slightly less than the inner diameter of the opening or aperture of the other chamber. The opening or aperture of one or both chambers may comprise (e.g., be lined) with a gasket (e.g., rubber, silicone, or other sealing material). In a particular embodiment, the opening or aperture of the first chamber and the opening or aperture of the second chamber form a male-female connector assembly (e.g., optionally with one or more gaskets between the connectors). The openings or apertures of the first and second chambers in the male-female connector assembly may be threaded such that the chambers may be screwed together (e.g., a Swagelock® connector).

The test compound may be in any gas and/or liquid within the chambers. For example, the test compound may be present in a gas and/or liquid carrier or solvent. In a particular embodiment, the test compound is contained within a liquid solvent within the first chamber. In a particular embodiment, the test compound is present in vapors within the first chamber. For example, the test compound may be present as vapors in an inert gas (e.g., N₂, noble gases) or air in the first chamber. Typically, the gas and/or liquid of the first chamber will be the same as the gas and/or liquid of the second chamber. However, the present invention encompasses methods wherein the gas and/or liquid of the first chamber is different than the gas and/or liquid of the second chamber. For example, the first chamber may comprise a gas and the second chamber may comprise a liquid or the first chamber may comprise a first gas and/or liquid and the second chamber may comprise a second (different) gas and/or liquid. Generally, the gas and/or liquid (e.g., carriers or solvents) used in the chambers are capable of traversing the test membrane.

The presence, amount, and/or concentration of the test compound in the second (receiving) chamber can be measured by any method. In certain embodiments, the mere presence of the test compound in the second chamber is detected. In certain embodiments, the amount and/or concentration of the test compound in the second chamber is measured. In a particular embodiment, the test compound is detected using a spectroscopy method (e.g., optical spectroscopy). Spectroscopic techniques for use with the instant invention include, without limitation, Raman spectroscopy (e.g., surface-enhanced Raman spectroscopy), infrared spectroscopy, Fourier-transform infrared spectroscopy (FTIR), gas chromatograph, gas chromatography-mass spectrometry (GC-MS), ion mobility spectrometry, air quality analyzers, ultraviolet-visible (UV-Vis) spectroscopy, and fluorescence spectroscopy. In a particular embodiment, the presence, amount, and/or concentration of the test compound in the second chamber is measured by Raman spectroscopy.

In certain embodiments, the presence, amount, and/or concentration of the test compound in the second chamber is measured more than once. As explained above, the methods of the instant invention allow for the real-time measurement of the movement of the test compound across a test membrane. In a particular embodiment, the presence, amount, and/or concentration of the test compound in the second chamber is measured at more than one timepoint. For example, the presence, amount, and/or concentration of the test compound in the second chamber can be measured at timepoint zero (e.g., when the test compound is introduced into the first chamber) and then at intervals (e.g., regular intervals) of time thereafter. The measurement of the test compound in the second chamber at different timepoints allows for the determination of the rate at which the test compound can traverse the test membrane. In a particular embodiment, the presence, amount, and/or concentration of the test compound in the second chamber is measured as a function of time.

The methods of the instant invention may also employ the use of nanoparticles, particularly metallic nanoparticles, within the permeation cell. In certain embodiments, the second chamber of the permeation cell comprises the nanoparticles in solution. In a particular embodiment, the methods of the instant invention further comprise adding nanoparticles to the second chamber. The presence of the nanoparticles in the second chamber (e.g., receiving chamber) can improve the signal to noise ratio during detection of the test compound, particularly by a spectroscopy method such as an optical spectroscopy method (e.g., Raman spectroscopy such as surface-enhanced Raman spectroscopy). Typically, the nanoparticles are less than 100 nm in diameter. For example, the nanoparticles may have a diameter of about 1 nm to about 100 nm, about 10 nm to about 90 nm, about 20 nm to about 80 nm, about 25 nm to about 75 nm, about 30 nm to about 70 nm, about 40 nm to about 60 nm, about 50 nm to about 60 nm, or about 45 nm to about 65 nm. Examples of metallic nanoparticles include, without limitation, silver nanoparticles, gold nanoparticles, and copper nanoparticles. In certain embodiments, the nanoparticles are selected from silver nanoparticles and gold nanoparticles.

In certain embodiments, the gas and/or liquid within the first and second chambers is static. In other words, the gas and/or liquid within the first and second chambers is not pumped into and/or removed from the chambers during the experiment. FIG. 2A provides a schematic of a permeation cell wherein the contents of the first and second chambers are static. The test compound present in the first chamber will traverse into the second membrane (e.g., by the principles of osmosis) unless the test membrane inhibits or prevents the traversal of the test compound.

Due to the extreme toxicity of certain test compounds, a permeation cell where the gas and/or liquid within the first and second chambers is static may be preferred. Indeed, the permeation cell may be disposable and/or intended for single time use. As such, the test compound needs only be inserted into the permeation cell once and then the permeation cell can be properly disposed of after the experiments.

As explained above, the first and second chambers may be contained within a permeation cell. In a particular embodiment, the permeation cell allows for the exchange of the test membrane with other test membranes. For example, the test membrane may be part of a replaceable cartridge that can be inserted in between the first and second chambers (e.g., seal (e.g., hermetically) the gap or space between the first and second chambers). The replaceable cartridge may seal (e.g., hermetically) to the opening or aperture of the first chamber and/or the opening or aperture of the second chamber (e.g., via a gasket or other seal). The exchange of test membranes allows for the rapid testing of different test membranes against the same or different test compounds with the same or different gas or liquid carriers/solvents.

The first and second chambers of the permeation cell may be of any shape. In a particular embodiment, the permeation cell is shaped or adapted to fit within the spectroscopy machine used to detect the test compound in the second chamber. In a particular embodiment, the first and second chambers are hemispherical. In a particular embodiment, the first and second chambers are cuboidal. In a particular embodiment, the first and second chambers are cylindrical. The first and second chambers may have the same shape and/or size (e.g., volume) or may differ in shape and/or size (e.g., volume).

The second chamber (and, optionally, the first chamber and/or the remainder of the permeation cell) is made of a material (e.g., glass or plastic) which does not impede the technique (e.g., spectroscopy technique) used to detect the test compound in the second chamber. The permeation cell may comprise an aperture, window, slot, hole, opening, or port to the second chamber to allow for the detection of the test compound in the second chamber by the desired technique (e.g., spectroscopy technique). In other words, the entirety of at least the second chamber is made of a material (e.g., glass or plastic) which does not impede the technique (e.g., spectroscopy technique) used to detect the test compound or at least a portion of the second chamber is made of a material (e.g., glass or plastic) which does not impede the technique (e.g., spectroscopy technique) used to detect the test compound.

As stated hereinabove, the first and second chambers may be of the same volume or may have different volumes. In certain embodiments, the second chamber (receiving chamber) is larger than the first chamber. For example, the second chamber may be about 2 times, 3 times, 4 times, or 5 times larger than the first chamber (e.g., by volume). In certain embodiments, the first and second chambers each hold less than about 10 ml in volume, particularly less than about 5 ml in volume. For example, each chamber may hold about 0.1 ml, 0.5 ml, 1.0 ml, 1.5 ml, 2.0 ml, 2.5 ml, or 3.0 ml. In a particular embodiment, the first chamber holds about 1.0 ml (e.g., +/−10%) and the second chamber holds about 2.0 to 3.0 ml (e.g., +/−10%).

In certain embodiments, the first and second chambers are contained within a disposable or single use permeation cell. In a particular embodiment, the permeation cell allows for the insertion of a test membrane. For example, the test membrane may be placed into a cartridge that can be inserted in between the first and second chambers wherein a seal (e.g., airtight and/or watertight) is formed such that the only way to traverse from the first chamber to the second chamber is through the test membrane. Alternatively, the first and second chambers may be separate units which can be assembled together with the test membrane positioned between the first and second chambers such that a seal (e.g., airtight and/or watertight; e.g., a hermetic seal) is formed such that the only way to traverse from the first chamber to the second chamber is through the test membrane. For example, one or both of the first and second chamber may have a gasket which can form a seal (e.g., airtight and/or watertight; e.g., a hermetic seal) upon the application of pressure (e.g., by tightening or screwing a connector (e.g., a Swagelock® connector)). The first and second chambers may be assembled together via clamps, screws, latches, or the like.

In certain embodiments, the disposable or single use permeation cell comprises a septum, aperture, opening, injection site, or port (e.g., injection port) in the first chamber and/or second chamber. The septum, aperture, opening, injection site, or port in the first and/or chamber is preferably resealable such that the contents of the first and/or chamber cannot escape. In certain embodiments, the first chamber of the permeation cell can be resealed after insertion of the test compound. In a particular embodiment, the septum, aperture, opening, injection site, or port is located at a distal or opposite position to the test membrane within the chamber. The septum, aperture, opening, injection site, or port of the second chamber may be used for injection (e.g., via syringe) of the carrier or solvent and, optionally, the nanoparticles. In a particular embodiment, the second chamber is filled carrier or solvent and, optionally, the nanoparticles by injection (e.g., via syringe) into the septum, aperture, opening, injection site, or port in the first chamber. The disposable or single use permeation cells of the instant invention may be preloaded or prefilled with the carrier or solvent and, optionally, the nanoparticles.

In certain embodiments, the gas or liquid within the first and/or second chambers of the permeation cell is not static. For example, the gas or liquid within the first chamber may be in connection (e.g., air or fluid connection) with a first circuit and/or the gas or liquid within the second chamber may be in connection (e.g., air or fluid connection) with a second circuit. The test compound will be present in the first chamber and first circuit and will only traverse into the second membrane and/or second circuit by traversing the test membrane.

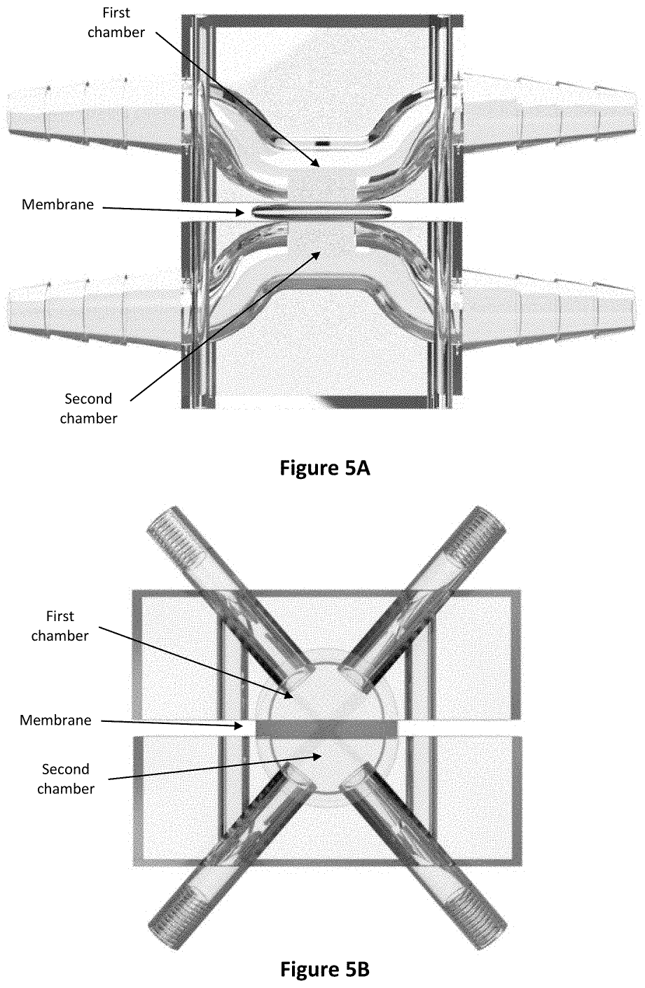

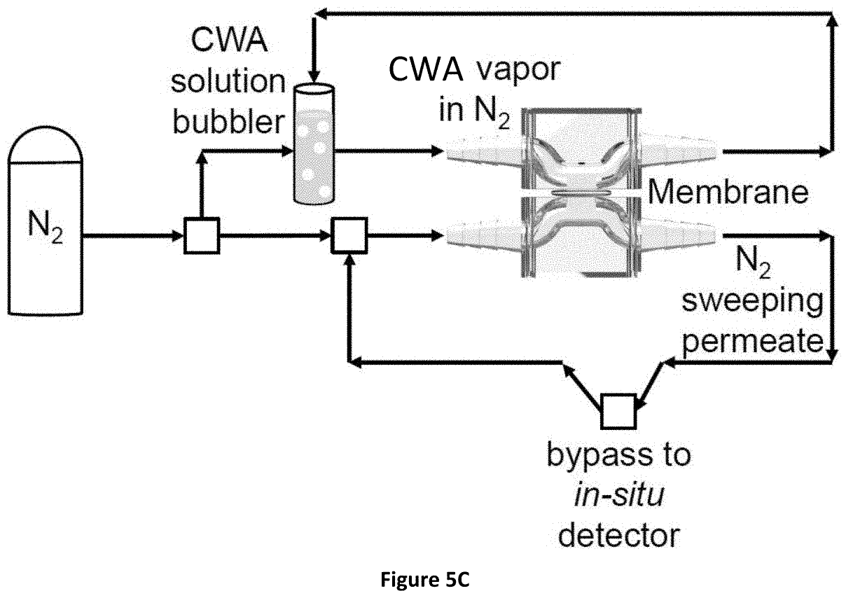

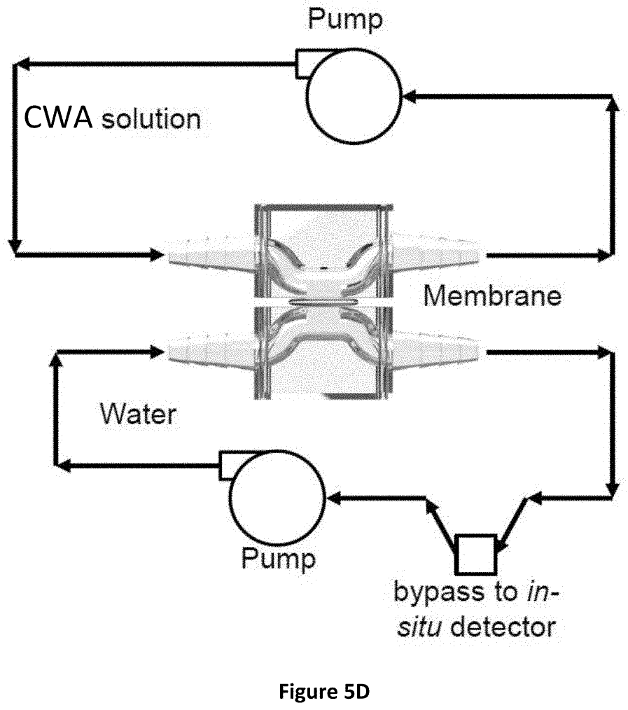

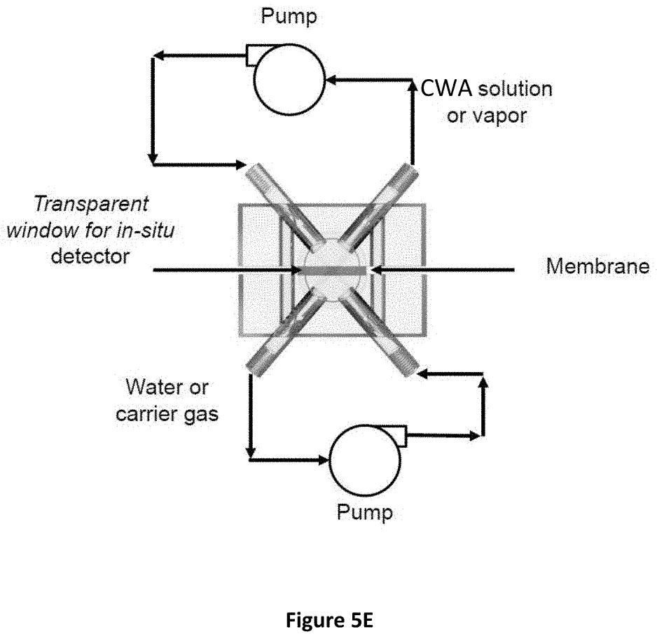

The permeation cell may comprise at least one inlet and at least one outlet to the first chamber and/or at least one inlet and at least one outlet to the second chamber. The inlets and outlets may be at any angle with regard to the target membrane. For example, the inlet may be directed at the target membrane or directed across the target membrane. FIG. 5A provides an example of a permeation cell comprising inlets and outlets. As seen in FIG. 5A, the permeation cell comprises a first chamber with an inlet and an outlet which allow for the easy connection of tubing or piping for creation of the first circuit. The permeation cell also comprises a second chamber with an inlet and outlet which allow for the easy connection of tubing or piping for creation of the second circuit. FIG. 5B also provides an example of a permeation cell comprising inlets and outlets. In contrast to the permeation cell of FIG. 5A, the inlets of the permeation cell of FIG. 5B are directed towards the test membrane as opposed to across the test membrane.

In certain embodiments, the methods of the instant invention use a gas phase permeation assembly. FIG. 5C provides a schematic of a gas-phase permeation assembly. The gas phase permeation assembly may comprise a permeation cell comprising at least one inlet and at least one outlet to the first chamber and/or at least one inlet and at least one outlet to the second chamber. The first chamber and the inlet and outlet to the first chamber form part of a first circuit. The first circuit may further comprise a solution bubbler or an aperture, opening, injection site, or port (e.g., injection port) by which a desired gas containing a test compound may be inserted. In a particular embodiment, the solution bubbler is a container comprising a solution of the test compound and an aperture, opening, injection site, or port (e.g., injection port) that allows the delivery of a gas, particularly air or an inert gas such as N₂. The first circuit may further comprise a pump. The second chamber and the inlet and outlet to the second chamber form part of a second circuit. The second circuit may further comprise an aperture, opening, injection site, or port (e.g., injection port) that allows the delivery of a gas, particularly air or an inert gas such as N₂. The second circuit may further comprise a pump. The second circuit may further comprise an in situ detector for detecting the presence of the test compound within the second circuit. Alternatively, as explained hereinabove, the presence of the test compound may be detected within the second chamber.

In certain embodiments, the methods of the instant invention use a liquid phase permeation assembly. FIGS. 5D and 5E provide schematics of a liquid-phase permeation assembly. The liquid phase permeation assembly may comprise a permeation cell comprising at least one inlet and at least one outlet to the first chamber and/or at least one inlet and at least one outlet to the second chamber. The first chamber and the inlet and outlet to the first chamber form part of a first circuit. The first circuit may further comprise a pump. The first circuit may further comprise an aperture, opening, injection site, or port (e.g., injection port) by which a desired liquid containing a test compound may be inserted. The second chamber and the inlet and outlet to the second chamber form part of a second circuit. The second circuit may further comprise a pump. The second circuit may further comprise an aperture, opening, injection site, or port (e.g., injection port) by which a desired liquid (e.g., the solvent in which the test compound is dissolved) may be inserted. The second circuit may further comprise an in situ detector for detecting the presence of the test compound within the second circuit. Alternatively, as explained hereinabove, the presence of the test compound may be detected within the second chamber. For example, FIG. 5E provides an aperture, window, slot, hole, opening, or port by which an in situ detector can be used to detect the presence of the test compound on the second chamber side of the test membrane.

The test membrane of the methods of the instant invention can be any membrane. Preferably, the test membrane is permeable to at least to air and/or water. In a particular embodiment, the test membrane is a polyelectrolyte membrane. Polyelectrolyte membranes may be composed of a polymer (e.g., a homopolymer or a copolymer (e.g., a diblock or triblock copolymer or an amphiphilic copolymer (e.g., amphiphilic block copolymer). Examples of polyelectrolyte membranes include, without limitation, sulfonated tetrafluoroethylene based fluoropolymer-copolymer (e.g., Nafion®) and sulfonated styrene-ethane/butadiene-styrene (sSEBS). In a particular embodiment, the test membrane comprises a metal catalyst. In a particular embodiment, the test membrane comprises a counterion. In a particular embodiment, the test membrane comprises a metal oxide and/or metal oxide nanoparticle. In a particular embodiment, the test membrane comprises a polyoxometalate.

As explained hereinabove, the test compound can be any chemical compound. Preferably, the chemical compound can be detected spectroscopically. In certain embodiments, the test compound is a toxin. In certain embodiments, the test compound is a chemical warfare agent. In a particular embodiment, chemical warfare agent is an agent classified as a schedule 1, 2, or 3 agent under the Chemical Weapons Convention of 1993. The chemical warfare agent may be in liquid form, gas form, solid form, or combinations thereof. The chemical warfare agent may be a nerve agent, blood agent, blister agent, pulmonary agent, incapacitating agent, and/or toxin. In a particular embodiment, the chemical warfare agent is a phosphororoganic. In a particular embodiment, the chemical warfare agent is a nerve agent. In a particular embodiment, the chemical warfare agent is a G-agent, H-agent, and/or V-agent. Examples of chemical warfare agents that are G-agents include, but are not limited to, tabun, sarin, soman, cyclosarin, ethyl sarin, 0-isopentyl sarin, 2-(dimethylamino)ethyl N,N-dimethylphosphoramidofluoridate, or a combination thereof. Examples of H-agents include, but are not limited to, sulfur mustard, 2-chloroethyl ethylsulfide (CEES; half mustard), or a combination thereof. Examples of V-agents include, but are not limited to, S-[2-(diethylamino)ethyl] O-ethyl ethylphosphonothioate, S-[2-(diethylamino)ethyl] O-ethyl methylphosphonothioate, 3-pyridyl 3,3,5-trimethylcyclohexyl methylphosphonate, 0-isobutyl S-(2-diethaminoethyl) methylphosphothioate, ethyl ({2-[bis(propan-2-yl)amino]ethyl} sulfanyl)(methyl) phosphinate (VX), 0,0-diethyl-S-[2-(diethylamino)ethyl] phosphorothioate, or a combination thereof. Examples of other chemical warfare agents include, but are not limited to, diisopropyl fluorophosphonate, dimethyl-methylphosphonate, malathion, or a combination thereof.

Because of their extreme toxicity, chemical warfare agents are often mimicked in experiments by simulants, that is, similar compounds of low toxicity whose chemistry and transport properties are close to those of chemical warfare agents. Accordingly, chemical warfare agent simulants may be used as the test compound. G-agents may be mimicked by alkylphosphonates and fluorophosphates (Frishman et al. (2000) Field Anal. Chem. Tech., 4:170-94; Suzin et al. (2000) Carbon 38:1129-33; Vo-Dinh et al. (1999) Field Anal. Chem. Tech., 3:346-56). Such compounds are strongly polar, such as, for example, malathion, a simulant for V-agents (Kosolapoff et al. (1954) J. Chem. Soc., 3222-25). Agents and simulant conformations and single-molecule properties are well-explored by ab initio modeling and spectroscopy, and forcefields of standard form having been developed for classical simulations (Suenram et al. (2004) J. Mol. Spectr., 224:176-84; Suenram et al. (2002) J. Mol. Spectr., 211:110-18; Walker et al. (2001) J. Mol. Spectr., 207:77-82; Kaczmarek et al. (2004) Struct. Chem., 15:517-25; Sokkalingam et al. (2009) J. Phys. Chem. B, 113:10292-97; Vishnyakov et al. (2011) J. Phys. Chem. A, 115:5201-09).

Test membranes may possess catalytic activity and chemically alter and/or cleave a test compound. Accordingly, the methods of the instant invention encompass detecting cleavage or breakdown products in the second chamber of the test compound provided in the first chamber.

The following example describes illustrative methods of practicing the instant invention and is not intended to limit the scope of the invention in any way.

### Example

### Materials and Methods

### Materials

Nafion®-117 ionomer membranes with an equivalent weight of 1100 g/SO₃H were purchased from Ion Power, Inc. (New Castle, Del.). Nexar® MD9200 solutions (11 wt. % in cyclohexane and heptane) were obtained from Kraton (Houston, Tex.). Salt reagents zinc nitrate hexahydrate (98%), and sodium hydroxide (>97%) were purchased from Sigma-Aldrich (St. Louis, Mo.).

### Solvent Casting of Nexar® Membranes

Nexar® films were solvent-casted from Nexar® MD9200 solutions (11 wt. % in cyclohexane and heptane) using a mixture of 100 mL of tetrahydrofuran (THF) and 30 mL of Nexar® MD9200 solution. The polymer solution was thoroughly mixed for 6 hours at room conditions. Afterwards, 25 mL of the solution was poured into 50 mL Teflon® evaporation dishes and the dishes were covered with P2 filter paper (to help slow down the evaporation rate of solvent) and were left overnight at room conditions. After 3 days, Nexar® films were slightly brown and about 500 μm in thickness and were qualitatively robust and flexible.

In Situ Growth of ZnO within Polyelectrolyte Membranes

Nexar® and Nafion® membranes were cut into 0.5 inch by 0.5 inch pieces and were immersed in HCl (1M) at room conditions for 1 hour to remove any impurities and then immersed in deionized (DI) water to remove any excess HCl from the previous step. Once washed, these membranes were labeled as “PEM-H+” where PEM represent either Nafion® or Nexar®. PEM-H+ membranes were immersed in 200 mL of 0.05M Zn(NO₃)₂ solutions at room conditions for 1 hour to allow for Zn²⁺ exchange and these samples were labeled “PEM-Zn²⁺”. Afterwards, PEM-Zn²⁺ membranes were rinsed in DI water and immersed in 200 mL of 0.5M NaOH at 60° C. for 1 hour. This step allows for Zn²⁺ to hydrolyze and form Zn(OH)₂. Membranes were rinsed again in DI water and blotted dry before putting the membranes in an oven at 100° C. for 1 hour. These samples were labeled “PEM-ZnO”.

### X-Ray Diffraction (XRD)

X-ray diffractograms were obtained using a Phillips XPert diffractometer (Bragg-Brentano geometry) with a CuKα anode (1.5405 Å). The instrument was operated at 45 kV and 40 mA in a continuous scanning mode at a 0.02°/step acquisition rate with a dwell time of 2 seconds/step from 20 angles over the range of 10° to 90° on a 20 scale using a 0.3 mm fixed receiving slit.

### Transmission Electron Microscopy (TEM)

Transmission electron micrographs were obtained by a JEOL 1200EX electron microscope with AMT-XR41 digital camera with an accelerating voltage of 80 kV and 2-seconds sample exposure time. Samples were supported on lacey carbon type-A 300 mesh copper grids.

### Spectroscopic Characterization

Raman spectra were acquired by using a Horiba LabRAM HR Evolution spectrometer with spectral resolution. The incident beam, which was a laser of 532 nm, and 80 mW initial power, was directed via a 90° macro lens of 40 mm focal length in the middle of the 2 ml vial that used to measure DMMP diffusion. The scattered light was collected through backscattering geometry and analysed with a 1800 gr/mm grating. Collection of the scattered light was achieved with an air-cooled open electrode 1024×256 pixels CCD at the temperature of −75° C. The acquisition time of each spectrum was 60-120 seconds, depending on the signal to noise (S/N) ratio and the number of accumulations varied in the range of 6-12. The spectral slit was fixed at 100 μm.

### Calibration of Raman Spectra

A calibration curve was developed for DMMP based on its peak located at 715 cm⁻¹ associated with the P—C stretching mode [(P—CH₃)] of DMMP. The range of 680 to 745 cm⁻¹ was used to calculate the mathematical area under the curve (AUC) at the aforementioned peak for the calibration curve.

### Results

Herein, a novel experimental setup is provided to measure via in situ Raman spectroscopy the membrane protective capabilities against harmful substances. As a practical example, the permeation of DMMP, a nerve agent simulant, through composite MONP-PEM membranes was studied. In situ and in operando Raman spectroscopy has become a powerful technique to study dynamic experiments, particularly those that require high sensitivity towards chemical and physical changes, such as changes in restructuring of metal-oxides during a chemical reaction (Elliott, et al., J. Catalysis (2019) 371:287-290; Tsilomelekis, et al., Catalysis Today (2010) 158(1-2):146-155), observing electrochemical changes in metal-oxides for charge storage applications (Chen, et al., Chem. Mater. (2015) 27(19):6608-6619), and monitoring reaction products during biomass conversion (Ramesh, et al., React. Chem. Eng. (2019) 4(2):273-277), as well as electrocatalytic water splitting reactions (Deng, et al., ACS Catalysis (2017) 7(11):7873-7889). Moreover, in situ Raman spectroscopy has been useful for monitoring liquid-phase kinetic phenomena such as functionalization of organic compounds for the production of urea-derivatives (Tireli, et al., Chem. Comm. (2015) 51(38):8058-8061), measuring concentrations of CO₂ within water at high pressures (Geng, et al., Fluid Phase Equilibr. (2017) 438:37-43), and real-time measurement of aqueous corrosion of borosilicate glass (Geisler, et al., Nature Mater. (2019) 18(4):342-348). In situ Raman spectroscopy measurements for permeation through membrane barriers have been also utilized for electrolytes (Yamanaka, et al., Electrochimica Acta (2017) 234:93-98; Yamanaka, et al., ChemSusChem (2017) 10(5):855-861).

Two sets of PEMs were used to assess their capabilities as protective materials, Nafion® and Nexar®. Nafion® membranes (DuPont) are part of a larger family of perfluorosulfonated ionomers (PFSI) that have been used in several applications such as proton exchange membrane fuel cells (PEMFCs) (Ketpang, et al., J. Membrane Sci. (2015) 488:154-165; Devrim, et al., Int. J. Hydrogen Energ. (2015) 40(44):15328-15335; Wehkamp, et al., Rsc Advances (2016) 6(29):24261-24266), super-acid catalysis (Ponomareva, et al., Applied Catalysis A: General (2019) 569:134-140; Liang, et al., Chem. Commun. (2015) 51(5):903-906), selective drying and humidification of gases (Peng, et al., Fuel (2019) 238:430-439; Schalenbach, et al., J. Phys. Chem. C (2015) 119(45):25145-25155), and chlor-alkali electrolysis (Hosseini, et al., Desalin. Water Treat. (2017) 84:299-308; Franco, et al., Appl. Sci-Basel (2019) 9(2):284). Nafion® shows great chemical stability to a vast amount of chemicals including very corrosive compounds such as hydrogen fluoride and hydrogen chloride gases. In addition, they present excellent mechanical and thermal stability thus making them ideal candidates for high temperature reactions in fuel cells (Matos, et al., Electrochimica Acta (2016) 196:110-117; Maiti, et al., Compos. Sci. Technol. (2018) 155:189-196) and particularly advantageous for chemical protection. Nafion® 117 has an ion exchange capacity (IEC) of 0.91 meq/g, which yields in average polymer networks with 1100 equivalent weight (EW).

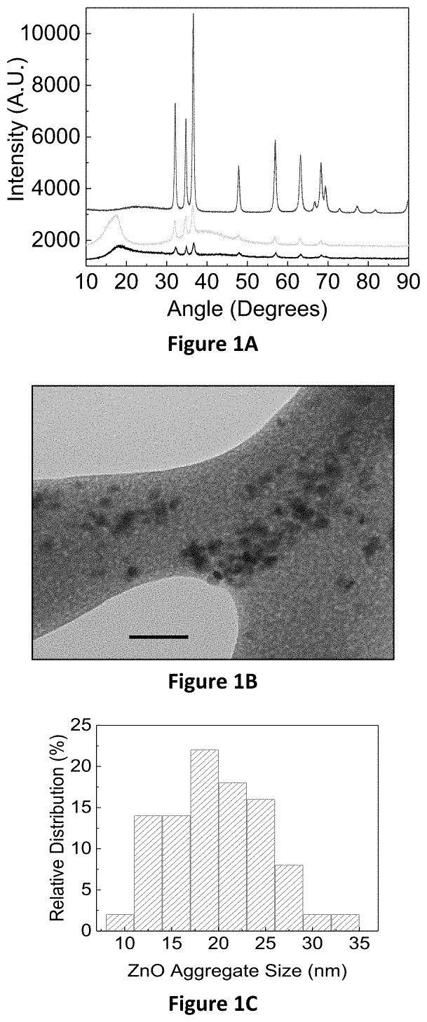

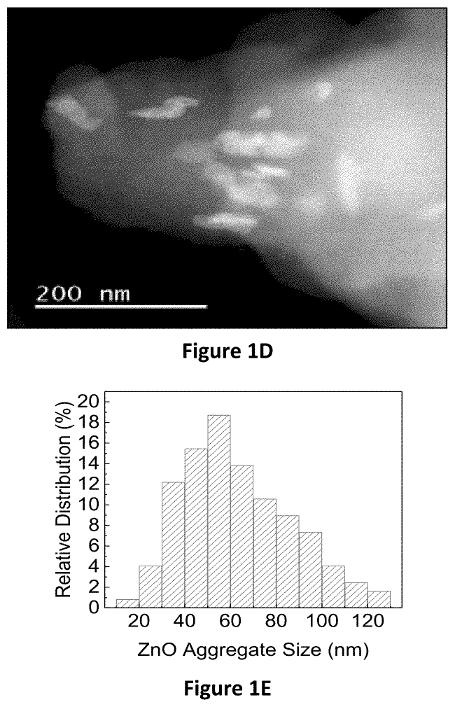

Nexar® MD9200 membranes (Kraton) are pentablock copolymers that have been used in several applications such as alcohol dehydration (Shi, et al., Sep. Purif. Technol. (2015) 140:13-22; Zuo, et al., ACS Appl. Mater. Interfaces (2014) 6(16):13874-83), water purification (Yeo, et al., Nanotechnology (2012) 23(24):245703), separation of solvents (Duong, et al., Environ. Sci. Technol. (2014) 48(8):4537-45), nanofiltration (Thong, et al., Environ. Sci. Technol. (2014) 48(23):13880-7), and fuel cells (Huang, et al., J. Membrane Sci. (2018) 545:1-10). These pentablock copolymers have been known to possess high water-vapor transmission rates, excellent mechanical and thermal stability (Akhtar, et al., J. Membrane Sci. (2019) 572:641-649) making them ideal for high performance clothing. Nexar® has an ion exchange capacity (IEC) of 2.0 meq/g, which yields in average polymer networks with 500 equivalent weight (EW). The in situ growth of MONP within PEM was performed as reported (Landers, et al., Angewandte Chemie-International Ed. (2016) 55(38):11522-11527). The samples were examined by x-ray diffraction (XRD) to confirm the presence of ZnO within the membranes. In FIG. 1A, one can observe that Nafion® and Nexar® do have the characteristic peaks of hexagonal lattice ZnO as they match the superposed peaks of reference bulk ZnO. Once the presence of ZnO was confirmed by XRD, transmission electron microscopy techniques were applied in an effort to image and quantify the size of the ZnO nanoclusters within the Nexar® substrates. FIGS. 1B, 1D present the ZnO nanoaggregates within PEM substrates. The ZnO nano-aggregate sizes range from 8-35 nm in Nafion®-ZnO samples (FIG. 1C). In the Nexar®-ZnO samples, larger nanoaggregates with a wider distribution are observed with ZnO aggregate size distributions that range from 10 to 120 nm (FIG. 1E). Although both PEM, Nafion® and Nexar® were subjected to the same conditions for the in situ growth of ZnO, there is a significant difference between the ZnO nanoaggregate size distributions.

The polymer microstructure plays an important role during the nucleation and growth of ZnO nanocrystals. These membranes nano-segregate in such a way that the hydrophilic subphases, containing the sulfonate ions, coalesce and form interconnected channels that allow for water and ion transport upon hydration (Vishnyakov, et al., J. Phys. Chem. B (2014) 118(38):11353-11364). Since the nano-crystalline ZnO grow within the hydrophilic subphases of the membranes, the growth of such crystals may be limited by the confinement of the hydrophilic domains and the limited supply of Zn²⁺ ions available, which allow for nano-sized crystals to grow without a capping agent. It has been demonstrated that by using organic solvents, growth of ZnO within membranes can be manipulated to favor the growth of particular crystalline planes within ZnO (Landers, et al., Angewandte Chemie-International Ed. (2016) 55(38):11522-11527). It should be noted, since the studied Nafion® and Nexar® membranes have different microstructures and degrees of sulfonation, these may lead to the difference in ZnO aggregate size distributions.

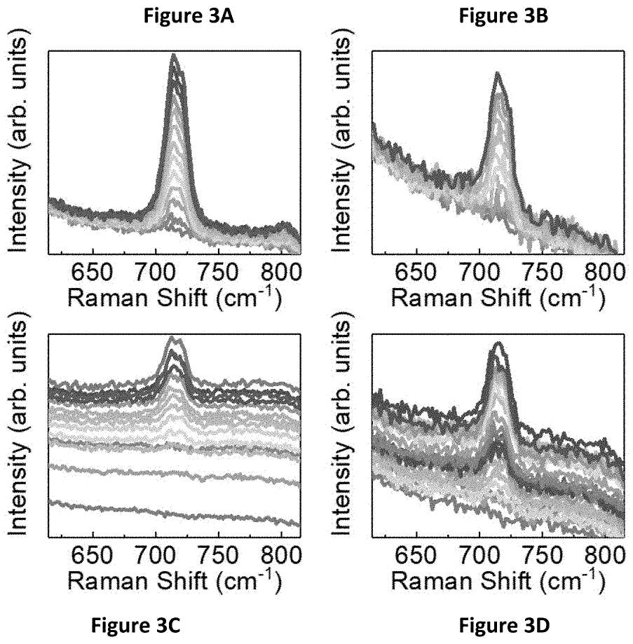

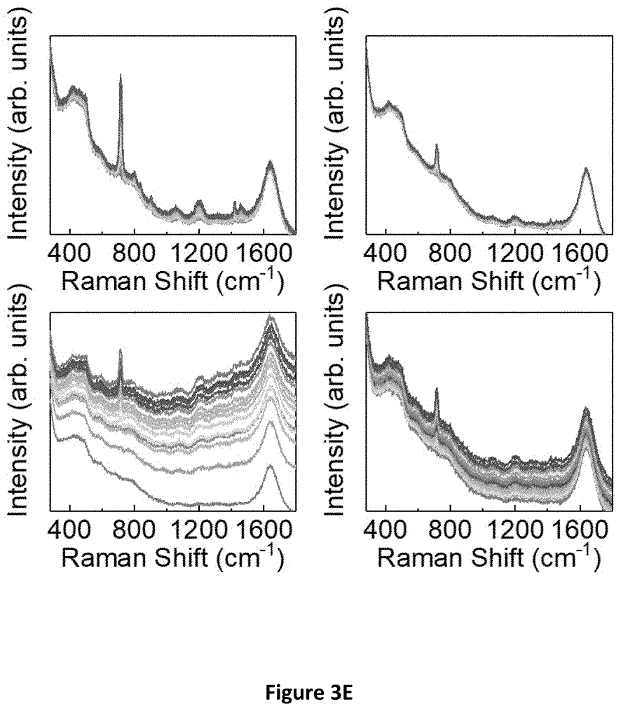

A customized permeation setup (FIG. 2A) was built to acquire in situ Raman measurements of DMMP permeation through the PEM and PEM-MONP composites. The initial configuration comprises a 12 mL vial (top vial—donor compartment) with 10 mL of a 10 vol. % solution of DMMP is connected to a 2 mL (bottom vial —receiving compartment) vial that filled by pure water. Both vials are separated by the PEM studied and were tightly sealed to avoid leakage from any compartment. 9 mm diameter samples of PEMs was utilized for all permeation experiments. A 532 nm laser is used to radiate the small vial with water to detect the progression of DMMP permeation in the receiving compartment as a function of time. FIG. 2B shows the calibration set of spectra used to calculate the area under the curve (AUC) of the DMMP peak located at 715 cm⁻¹, which is associated with its P—C stretching mode [(P—CH₃)] (Ruiz-Pesante, et al., In Detection of Simulants and Degradation Products of Chemical Warfare Agents by Vibrational Spectroscopy, Defense and Security Symposium, SPIE (2007) p. 10). FIG. 2C shows a linear correlation with an R² of 0.999 over the entire range of DMMP concentrations studied. FIG. 3 shows the evolution of the DMMP peak located at 715 cm⁻¹ as a function of time, for full DMMP spectra see FIG. 3E. As it can be observed, the intensity of the peak at 715 cm⁻¹ after one hour of permeation are most intense in the membranes without nanoparticles, Nafion®-H⁺ and Nexar®-H⁺, and is the peak intensity is significantly less with the ZnO incorporated membranes. It is evident that the incorporation of nanoparticles does have a great influence over the permeation rate of DMMP across the PEM, as it is seen on both Nafion® and Nexar® cases. It is possible that the additional ZnO-DMMP may hinder DMMP diffusion as ZnO serves as attractive centers from which DMMP may be strongly adsorbed to DMMP strongly adsorbs to metal-oxide surfaces (Henych, et al., Arabian J. Chem. (2016) dx.doi.org/10.1016/j.arabjc.2016.06.002). The reduced signal/noise ratio in some of our measurements is due to the combination of the low concentration as well as fast acquisition time. After performing a background subtraction and peak fitting for each spectra the AUC is calculated and later concentrations of permeated DMMP as a function of time are extracted from the calibration curve in FIG. 2C.

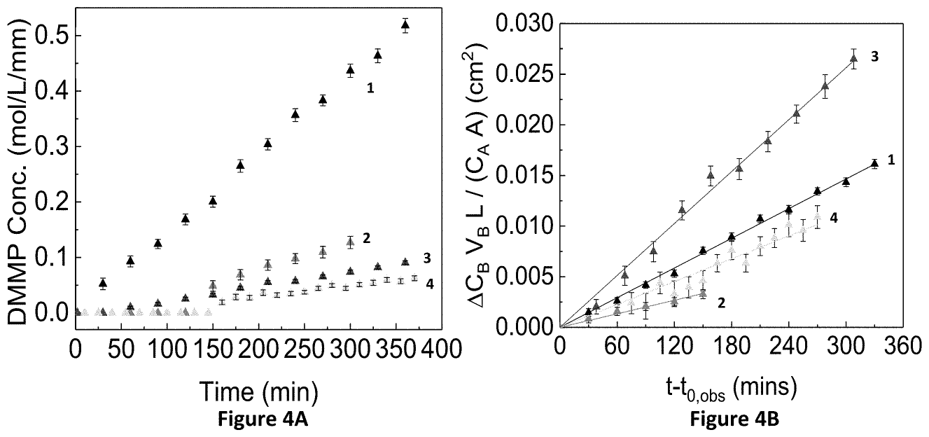

When plotting the permeated DMMP concentration on the receiving compartment as a function of time, as in FIG. 4A, it can be seen that Nafion®-H⁺ has an observable breakthrough time, t_{0,obs}, defined as the time of the first observation of the non-zero concentration of DMMP in the receiving compartment, of 15 minutes when compared to Nafion®-ZnO, which has a breakthrough time of 135 min, an order of magnitude longer. A similar increase of the breakthrough time upon addition of ZnO is found for Nexar® samples, 45 and 145 minutes for Nexar®-H⁺ and Nexar®-ZnO, respectively. In addition, the permeation rate of Nexar®-H⁺ is approximately 1.8, 2.3, and 3.8 times faster than Nafion®-H⁺, Nexar®-ZnO, and Nafion-ZnO, respectively. These results show that the incorporation of ZnO nanoparticles has a large impact on both, breakthrough time and permeation rate in Nafion® and Nexar® samples, which demonstrates the enhancement in the protective capabilities that PEM-ZnO have against DMMP. FIG. 4A shows that DMMP permeation through PEM can be analyzed by using a linear equation for diffusion in a plane sheet geometry (Crank, J., The Mathematics of Diffusion. 2 ed.; Oxford University Press, 1975):

C_{B}(t)=[(PC_{A}A)/(V_{B}L)]t (1)

Equation 1 implies that C_{A}>>C_{B}, where C_{A} and C_{B} are the DMMP concentrations in the donating and receiving compartments, respectively. L is the membrane thickness, A is the cross-sectional area of the membrane, and P is the permeability coefficient for DMMP. DMMP permeability, P is defined as the product DK, where D is the DMMP diffusion coefficient, and K is the partition coefficient (ratio of DMMP concentrations inside membrane and in the bulk solution at equilibrium).

Data shown in FIG. 4A could be fit to by using Equation 1 and transport properties could be extracted. To compare membrane permeabilities across multiple samples, one has to account for the difference in the membrane thickness, approximately 190 μm for Nafion® and 500 μm for Nexar®. Rearranging Equation 1 into the following form below in Equation 2:

(ΔC_{B}(t)V_{B}L)/(C_{A}A)=P(t−t_{0,obs}) (2),

provides a direct comparison between the sample permeabilities. Equation 2 presents the data shifted to the point, t_{0,obs}, of the first observation of a non-zero DMMP concentration in the receiving compartment and ΔC_{B}(t)=C_{B}(t)−C_{B}(t_{0,obs}). The observed breakthrough times, t_{0,obs}, were obtained from FIG. 4A. The permeation data in the shifted coordinates of Equation 2 is shown in FIG. 4B. The slopes of the linear fits of permeation curves in FIG. 4B give the permeability constants, P. Linear fits of permeation curves in FIG. 4B had excellent agreement with an R² of 0.99, for the exception of Nexar®-H⁺, which had an R² of 0.98. Obtained transport parameters are given in Table 1.

TABLE 1
List of transport properties of DMMP across PEM studied.

| | P (cm²/s) × 10⁷ | t_{0, obs}^{a} (min) |
|---|---|---|
| Nafion ®-H⁺ | 8.14 | 15 |
| Nafion ®-ZnO | 3.72 | 135 |
| Nexar ®-H⁺ | 14.20 | 45 |
| Nexar ®-H⁺ | 6.27 | 145 |

^{a}Measurements have an uncertainty of ±15 minutes.

A novel in situ Raman spectroscopy setup is built to measure agent permeation across protective barriers and applied to study DMMP permeation through ZnO loaded Nafion® and Nexar® polyelectrolyte membranes. It is shown that, incorporation of ZnO nanoparticles increases the breakthrough time and reduces the agent permeability, providing better protective capabilities of composite membranes. Of all samples studied, Nafion®-ZnO seems to have the best protective capabilities against DMMP permeation.

A number of patent and non-patent publications may be cited herein in order to describe the state of the art to which this invention pertains. The entire disclosure of each of these publications is incorporated by reference herein.

While certain embodiments of the present invention have been described and/or exemplified above, various other embodiments will be apparent to those skilled in the art from the foregoing disclosure. The present invention is, therefore, not limited to the particular embodiments described and/or exemplified, but is capable of considerable variation and modification without departure from the scope and spirit of the appended claims.

Moreover, as used herein, the term “about” means that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

What is claimed is:

1. A method for the detection of the permeation of a test compound across a membrane, said method comprising:
a) adding said test compound to a first chamber of a permeation cell, wherein said permeation cell comprises a first chamber and a second chamber, wherein said first chamber and second chamber are separated by said membrane, and wherein said first chamber and a second chamber are in gas or liquid communication across the test membrane; and
b) in situ detecting the presence of said test compound in the second chamber by a spectroscopic method,
wherein an increase in the presence of said test compound in the second chamber indicates that said membrane is permeable to said test compound.

2. The method of claim 1, wherein said spectroscopic method is selected from the group consisting of Raman spectroscopy, infrared spectroscopy, Fourier-transform infrared spectroscopy (FTIR), gas chromatograph, gas chromatography-mass spectrometry (GC-MS), ion mobility spectrometry, air quality analyzers, ultraviolet-visible (UV-Vis) spectroscopy, and fluorescence spectroscopy.

3. The method of claim 2, wherein said spectroscopic method is Raman spectroscopy.

4. The method of claim 1, wherein step b) comprises quantifying the concentration of the test compound in the second chamber and/or detecting the presence of the test compound over time.

5. The method of claim 1, wherein said test compound is in a liquid solvent.

6. The method of claim 1, wherein said test compound is in a vapor of an inert gas.

7. The method of claim 1, wherein said test compound is a toxin.

8. The method of claim 1, wherein said test compound is a chemical warfare agent or a simulant thereof.

9. The method of claim 8, wherein the chemical warfare agent comprises a G-agent, an H-agent, a V-agent, or a combination thereof.

10. The method of claim 9, wherein the H-agent comprises sulfur mustard, 2-chloroethyl ethylsulfide (CEES), or a combination thereof.

11. The method of claim 9, wherein the V-agent comprises S-[2-(diethylamino)ethyl] O-ethyl ethylphosphonothioate, S-[2-(diethylamino)ethyl] O-ethyl methylphosphonothioate, 3-pyridyl 3,3,5-trimethylcyclohexyl methylphosphonate, O-isobutyl S-(2-diethaminoethyl) methylphosphothioate, Ethyl ({2-[bis(propan-2-yl)amino]ethyl}sulfanyl)(methyl)phosphinate (VX), O,O-diethyl-S-[2-(diethylamino)ethyl] phosphorothioate, or a combination thereof.

12. The method of claim 9, wherein the G-agent comprises tabun, sarin, soman, cyclosarin, ethyl sarin, O-isopentyl sarin, 2-(dimethylamino)ethyl N,N-dimethylphosphoramidofluoridate, or a combination thereof.

13. The method of claim 8, wherein the chemical warfare agent comprises, diisopropyl fluorophosphonate, dimethyl-methylphosphonate, malathion, or a combination thereof.

14. The method of claim 1, wherein said first chamber comprises at least one inlet or port by which a gas or liquid may be inserted.

15. The method of claim 1, wherein said second chamber comprises metallic nanoparticles.

16. The method of claim 15, wherein said metallic nanoparticles are selected from the group consisting of copper nanoparticles, gold nanoparticles, and silver nanoparticles.
